(19) United States
(12) Patent Application Publication
(10) Pub. No.: US 2022/0347351 A1
(43) Pub. Date: Nov. 3, 2022

(54) NOVEL POROUS SCAFFOLD AND METHOD FOR MANUFACTURING SAME

(71) Applicant: PLCOSKIN CO., LTD., Seoul (KR)

(72) Inventors: Wooyeol Baek, Seoul (KR); Tai-Suk Roh, Seoul (KR); Won-Jaei Lee, Seoul (KR)

(21) Appl. No.: 17/764,513

(22) PCT Filed: Oct. 8, 2020
(86) PCT No.: PCT/KR2020/013786

§ 371 Date: Mar. 28, 2022

(30) Foreign Application Priority Data
Oct. 8, 2019 (KR) 10-2020-0129849
Oct. 10, 2019 (KR) 10-2019-0125136

(51) Int. Cl. A61L 27/56; A61L 27/18; A61L 27/34; B33Y 80/00; B33Y 40/20; B29C 71/04

(52) U.S. Cl. CPC A61L 27/56; A61L 27/18; A61L 27/34; B33Y 80/00; B33Y 40/20; B29C 71/04; A61L 2430/10; A61L 2430/22; A61L 2420/02; B29K 2995/0056; B29L 2031/753

(57) ABSTRACT

The present invention relates to a porous scaffold having excellent tissue engineering properties, and a method for manufacturing same. The scaffold of the present invention can be manufactured by a simple process, and exhibits high tensile strength and biocompatibility, as well as an excellent cell engraftment rate, and thus can be useful as a support composition for various of human transplantation, for example, as a support for artificial ligaments or abdominal wall reinforcement.

### CROSS-REFERENCE TO RELATED APPLICATIONS

This application is a U.S. National Stage entry of International Patent Application no. PCT/KR2020/013786, filed Oct. 8, 2020, which claims the benefit of priority of Korean Patent Applications nos. 10-2020-0129849, filed Oct. 8, 2020, and 10-2019-00125136, filed Oct. 10, 2019.

### TECHNICAL FIELD

The present disclosure relates to a biocompatible porous scaffold, a support composition for human body transplantation comprising the same, and a method for preparing the same.

### BACKGROUND ART

Recently, as the fields of bionics, material engineering, and surgical operation have been greatly developed, tissue engineering for the purpose of replacing and regenerating lost body tissues is making remarkable progress. Tissue engineering aims to understand the correlation between the structure and function of biological tissues by combining life science, engineering, and medicine, and it aims to maintain, improve, or restore body functions through artificial tissues that can be transplanted in the body in order to replace with normal tissues or regenerate damaged tissues or organs based on this.

The loss of body tissues is due to various causes such as degenerative diseases, trauma, surgical removal of tumors, and certain congenital malformations, and the body tissues can be restored to the original state only through the regeneration of irreversibly lost tissues. In order to induce regeneration of lost tissues through tissue engineering, it is important to first prepare a biodegradable polymer support (scaffold) similar to biological tissues. The main requirement of the support material used for the regeneration of human body tissues is that the tissue cells should sufficiently play a role of a substrate or frame so that the tissue cells may adhere to the material surface to form a tissue with a three-dimensional structure, and they should also be able to play a role of an intermediate barrier located between the transplanted cells and the patient cells.

After the scaffold is transplanted into a subject, when engraftment of cells necessary for tissue regeneration is induced, and the formation of new tissues is initiated, the newly formed tissues should fill the space as they disappear over time. Therefore, it is preferable that the scaffold is a biodegradable one that does not require surgical removal, and the scaffold should not cause immunorejection, inflammatory response, or long-term fibrous encapsulation, should not undergo shrinkage of the graft volume, and should be free from serious complications such as prosthetic implants.

Therefore, after serving as a physical support for an appropriate period, the scaffold should have a certain level of mechanical strength and elastic force along with biodegradability in order to efficiently induce tissue reformation while disappearing naturally. Therefore, for this purpose, it is very important to select a structure that is the most suitable for the most suitable natural or synthetic polymer.

Numerous papers and patent documents are referenced throughout the present specification and their citations are indicated. The disclosure contents of the cited papers and patent documents are inserted into the present specification by reference in their entirety to more clearly describe the level of the technical field to which the present disclosure pertains and the content of the present disclosure.

### DISCLOSURE

### Technical Problem

The present inventors have made intensive research efforts in order to develop a scaffold for efficient tissue regeneration that can be prepared by a relatively simple process while having sufficient physical strength and excellent biocompatibility. As a result, the present inventors have discovered that, when a mesh-type support composed of strands each having a certain diameter while having pores each having a certain size is prepared with a first polymer, and then the surface of the mesh-type support is coated with a second polymer having biocompatibility as a polymer different from the first polymer, the mesh-type support coated with the second polymer can be used as scaffolds for human body transplantation for various uses, including artificial ligaments and supports for reinforcing the abdominal wall, by showing not only high tensile strength and biocompatibility, but also a remarkably excellent cell engraftment rate.

Further, the present inventors have completed the present disclosure by discovering the fact that, when two biocompatible polymers with different structures and functions are manufactured into a three-dimensional porous structure and a two-dimensional porous structure respectively, and then joined, remarkably improved physical properties are exhibited while maintaining the unique functions such as tissue regeneration, wound healing, provision of in vivo binding force, and the like.

Accordingly, an object of the present disclosure is to provide a porous scaffold and a method for preparing the same.

Another object of the present disclosure is to provide a support for human body transplantation including the porous scaffold.

Other objects and advantages of the present disclosure will become more apparent from the following detailed description of the invention, claims, and drawings.

### Technical Solution

According to an aspect of the present disclosure, the present disclosure provides a method for preparing a porous scaffold, comprising:

(a) producing a polymer mesh having pores with an area of 0.1 to 0.5 mm² and strands each having a diameter of 0.1 to 0.3 mm from a solution of a first polymer; and

(b) coating the surface of the produced polymer mesh with a solution of a second polymer having biocompatibility.

The present inventors have made intensive research efforts in order to develop a scaffold for efficient tissue regeneration that can be prepared by a relatively simple process while having sufficient physical strength and excellent biocompatibility. As a result, the present inventors have discovered that, when a mesh composed of strands each having a diameter of 0.1 to 0.3 mm while having pores of 0.1 to 0.5 mm² is prepared with a first polymer, and then the surface of the mesh is coated with a second polymer having biocompatibility as a polymer different from the first polymer, the mesh coated with the second polymer can be used as scaffolds for human body transplantation for various uses, including artificial ligaments and supports for reinforcing the abdominal wall, by showing not only high tensile strength and biocompatibility, but also a remarkably excellent cell engraftment rate.

The term “scaffold” in the present specification refers to a tissue engineering structure for promoting recovery and regeneration of damaged tissues by attaching living cells, specifically, cells derived from damaged tissues or cells involved in the recovery of the damaged tissues. The term “cell attachment” indicates that cells are directly or indirectly adsorbed to a matrix or other cells while maintaining their intrinsic biological activities. Specifically, the scaffold according to the present disclosure may have a planar structure consisting of a single mesh or a three-dimensional structure in which a plurality of meshes are stacked.

The term “polymer” in the present specification refers to a synthetic or natural high molecular compound in which the same or different types of monomers are continuously combined. Thus, examples of the polymer include homopolymers (polymers in which one type of monomer is polymerized) and interpolymers prepared by the polymerization of at least two different monomers, and examples of the interpolymers include both copolymers (polymers prepared from two different monomers) and polymers prepared from more than two different monomers.

According to a specific embodiment of the present disclosure, the first polymer used in the present disclosure is selected from the group consisting of polycaprolactone (PCL), poly(L-lactic acid) (PLLA), poly(glycolic acid) (PGA), poly(lactic-co-glycolic acid) (PLGA), poly(L-lactide-co-ε-caprolactone) (LCL), and combinations thereof

More specifically, the first polymer is polycaprolactone (PCL).

According to the present disclosure, the first polymer forms a mesh having pores each having a certain size while strands each having a certain thickness at regular intervals intersect. In order to efficiently achieve recovery and regeneration of damaged tissues which are the ultimate object of the present disclosure, the pores should have the most suitable size in terms of the mechanical strength and elastic force of the mesh itself as well as attachment, proliferation, and activity maintenance of cells, and induction of new blood vessels during the tissue regeneration. Accordingly, the suitable pore area is specifically 0.1 to 0.5 mm², more specifically 0.1 to 0.4 mm², still more specifically 0.2 to 0.3 mm², and most specifically about 0.25 mm².

The term “pore area” in the present specification refers to the average area of repeated pores appearing through the intersection of strands in the mesh structure according to the present disclosure prepared with the first polymer, and such an area refers to the area measured before performing coating using a second polymer solution to be described later.

Moreover, the diameter of the strand forming the mesh together with the above-described pore area is important in order to secure physical properties suitable as a support for human body transplantation by having appropriate elastic modulus and tensile modulus. Accordingly, the strand has a suitable diameter of specifically 0.1 to 0.3 mm, more specifically 0.15 to 0.25 mm, and most specifically about 0.2 mm.

The step of producing the polymer mesh from the first polymer solution according to the present disclosure may use various methods known in the art, and examples of the methods include a three-dimensional printing method, a solvent-casting particulate leaching method, a gas foaming method, a fiber mesh/fiber bonding method, a phase separation method, a melt molding method, a freeze drying method, and an electrospinning method, but are not limited thereto.

According to the present disclosure, the scaffold has biocompatibility, in addition to the mechanical strength described above, by coating a polymer mesh, prepared with a first polymer solution, with a second polymer solution having biocompatibility.

The term “biocompatibility” in the present specification refers to properties that do not cause short-term or long-term side effects when administered in vivo and in contact with cells, tissues or body fluids of organs, and specifically, refers to tissue compatibility and blood compatibility that do not cause tissue necrosis or coagulate blood in contact with biological tissues or blood, as well as biodegradability that disappears after a certain period of time after administration in vivo.

The term “biodegradability” in the present specification refers to properties of being naturally decomposed when exposed to a physiological solution of pH 6 to 8, and specifically, refers to properties capable of being decomposed according to the passage of time by body fluids in a living body, degrading enzymes, or microorganisms. A biodegradable polymer usable in the present disclosure may also be any synthetic or natural polymer as long as it is a polymer having the above-described biodegradability, and examples thereof include collagen, gelatin, chitosan, hyaluronic acid, poly(valerolactone), poly(hydroxy butylate), poly(hydroxyvalerate), and combinations thereof, but are not limited thereto.

According to a specific embodiment of the present disclosure, the second polymer having biocompatibility is a natural polymer, more specifically collagen, and most specifically type 1 collagen.

According to a more specific embodiment of the present disclosure, the collagen solution is used at a concentration of 0.2 to 0.8% (v/v), more specifically 0.3 to 0.7% (v/v), and most specifically 0.4 to 0.6% (v/v).

The term “coating” in the present specification refers to forming a new layer having a certain thickness by modifying a specific material on the target surface, and the target surface and the coating material may be modified through an ionic bond or noncovalent bond. The term “noncovalent bond” is a concept including bonds generated by acting interactions such as hydrogen bonds and van der Waals bonds alone or together with the physical bonds as well as physical bonds such as adsorption, cohesion, entanglement, and entrapment. In the present disclosure, when the polymer mesh is coated with the second polymer solution, a sealed layer may be formed while completely surrounding the surface of the mesh, or a partially sealed layer may be formed.

According to a specific embodiment of the present disclosure, the method according to the present disclosure further comprises performing plasma treatment on a surface of the polymer mesh between the step (a) and the step (b).

According to the present disclosure, when a polymer mesh is prepared using a hydrophobic polymer such as polycaprolactone (PCL) as the first polymer, the polymer mesh may be homogeneously coated with a hydrophilic second polymer having biocompatibility through a pretreatment process that imparts hydrophilicity to a hydrophobic mesh. When plasma discharge is applied to the surface of a polymer material, as gas-reactive species are formed, the hydrophilicity of the surface increases through reaction with the polymer surface layer and cleavage of elemental bonds through energy transfer.

Specifically, plasma treatment may be performed under medium vacuum conditions of 1.0 to 0.1 Torr at room temperature.

Specifically, the plasma surface treatment is performed for 45 to 90 seconds, more specifically 50 to 80 seconds, and most specifically 50 to 70 seconds.

As shown in Examples to be described later, when plasma treatment is performed for 45 seconds or more, the hydrophilicity of the surface is increased so that a uniform collagen film is formed with almost no air bubbles generated on the surface of the mesh. However, when plasma treatment is performed for more than 90 seconds, there is a disadvantage in that the molecular weight is reduced from the surface of the first polymer so that the mechanical strength is weakened.

According to another aspect of the present disclosure, the present disclosure provides a porous scaffold comprising:

(a) a first polymer mesh having pores with an area of 0.1 to 0.5 mm² and strands each having a diameter of 0.1 to 0.3 mm; and

(b) a second polymer having biocompatibility with which the surface of the first polymer mesh is coated.

Since the first polymer and the second polymer used in the present disclosure have already been described above, their descriptions are omitted in order to avoid excessive overlapping.

According to still another aspect of the present disclosure, the present disclosure provides a support composition for human body transplantation comprising the porous scaffold.

The term “transplantation” in the present specification refers to a process of delivering biological tissues, cells, or an artificial support that accommodates the biological tissues and cells from a donor to a recipient for the purpose of maintaining the functional integrity of the tissues or cells transplanted into the recipient. Accordingly, the term “support for transplantation” refers to a physical support used in the process of delivering the biological tissues or cells to the recipient.

According to a specific embodiment of the present disclosure, the support composition according to the present disclosure is a support composition used for ligament reconstruction, craniofacial reconstruction, maxillofacial reconstruction, tissue reconstruction after removal of melanoma or head and neck cancer, chest wall reconstruction, delayed burn reconstruction, pelvic reinforcement, genital reinforcement, or abdominal wall reinforcement, and more specifically a support composition used for ligament reconstruction or abdominal wall reinforcement.

According to another aspect of the present disclosure, the present disclosure provides a method for tissue reconstruction comprising transplanting the above-described support composition according to the present disclosure in vivo.

According to another aspect of the present disclosure, the present disclosure provides a method for preparing a dual structure porous scaffold, comprising embossing a first polymer having biocompatibility into a mesh form on the surface of a support containing a second polymer having biocompatibility.

The present inventors have completed the present disclosure by discovering the fact that, when two biocompatible polymers with different structures and functions are manufactured into a three-dimensional porous structure and a two-dimensional porous structure respectively, and then joined, remarkably improved physical properties are exhibited while maintaining the unique functions of the respective polymers such as tissue regeneration, wound healing, provision of binding force, and the like.

Since the second polymer used in the present disclosure has already been described above, the description thereof is omitted in order to avoid excessive overlap. The second polymer having biocompatibility according to the present disclosure may be collagen, and in this case, the support containing the second polymer may be a collagen sponge.

The term “sponge” in the present specification refers to a spongy porous material which is composed of a three-dimensional network connected by an ionic or covalent bond of polymers and uses water as a dispersion medium. The collagen sponge according to the present disclosure may be used without limitation as long as it is a spongy structure having voids or pores in collagen, and for example, it may be prepared by freeze-drying a collagen solution or dispersion, or various commercially available ready-made collagen sponges may be purchased and used.

Since the first polymer used in the present disclosure has also been described above, the description thereof is omitted in order to avoid excessive overlap. Specifically, the first polymer according to the present disclosure may be polycaprolactone (PCL).

The dual structure porous scaffold according to the present disclosure may be fabricated as a conjugate of a collagen sponge-PCL mesh by embossing a first polymer, for example, PCL into a mesh form on a second polymer-containing support, for example, the surface of a collagen sponge. The term “embossing” in the present specification refers to a process of bonding PCL polymer to the sponge surface so that a mesh form is engraved on the surface of the collagen sponge in a protruding form.

According to a specific embodiment of the present disclosure, the embossing may be performed by outputting the first polymer in a mesh form using a three-dimensional printer on the surface of the second polymer-containing support.

According to a specific embodiment of the present disclosure, the mesh form includes strands each having a diameter of 0.3 to 0.5 mm and a spacing between the strands of 0.1 to 0.3 mm.

According to another aspect of the present disclosure, the present disclosure provides a dual structure porous scaffold including the following:

(a) a support containing a second polymer having biocompatibility; and

(b) a first polymer mesh which is bonded to the surface of the support and has biocompatibility.

Since the process of bonding the first polymer mesh to the second polymer-containing support using a first polymer, a second polymer, a support, and embossing which are used in the present disclosure has already been described above, the description thereof will be omitted in order to avoid excessive overlap.

The dual structure porous scaffold according to the present disclosure (for example, a collagen sponge-PCL mesh conjugate) is excellent in tensile strength and bonding strength compared to general collagen sponges used for regenerative treatment of bone tissues, skin tissues, etc. and has biodegradable properties, thereby providing a more stable bonding function and a remarkably improved fixation function within the human body for the period required for wound healing and tissue regeneration.

### Advantageous Effects

The features and advantages of the present disclosure are summarized as follows:

(a) the present disclosure provides a porous scaffold having excellent tissue engineering characteristics and a method for preparing the same; and

(b) the scaffold according to the present disclosure not only can be prepared by a simple process, but also can exhibit a remarkably excellent cell engraftment rate as well as high tensile strength and biocompatibility so that it can be usefully used as a support composition for human body transplantation of various uses, including artificial ligaments and supports for reinforcing the abdominal wall.

### BRIEF DESCRIPTION OF DRAWINGS

FIG. 1 shows the results of observing a polymer mesh according to the present disclosure prepared using a three-dimensional printer with an optical microscope.

FIG. 2 is optical photographs showing air bubbles on the surface of the polymer mesh generated after the polymer mesh according to the present disclosure is subjected to plasma surface treatment for various time periods and then coated with collagen.

FIG. 3 is a picture showing the macroscopic shapes of the collagen-coated meshes.

FIG. 4 is electron micrographs showing the microscopic shapes of the collagen-coated meshes.

FIG. 5 is a drawing showing the results of analyzing the physical strength values of a collagen-coated mesh for transplantation and acellular allogeneic dermis.

FIGS. 6A and 6B show the results of analyzing elements present on the surfaces of a mesh which is not coated with collagen (FIG. 6A) and a mesh which is coated with 0.5% collagen (FIG. 6B) using Energy Dispersive X-Ray Spectroscopy (EDS) (EDAX, USA).

FIGS. 7A and 7B show the results of observing active cells after cell culture (FIG. 7A) and the results of quantifying the observation results (FIG. 7B) in order to compare the cellular reactivities of the meshes depending on whether or not the meshes are coated with collagen.

FIGS. 8A, 8B and 8C show the results of staining the collected tissues with Masson's Trichrome by collecting tissues after transplanting the meshes into the acellular allogeneic dermis and the experimental animal dermis for 6, 12, and 20 weeks respectively in order to verify the biological safety of the meshes depending on whether or not the meshes are coated with collagen (FIG. 8A), and based on the staining results, shows the results of quantifying the thickness values of the films according to the inflammatory reaction (FIG. 8B) and the thickness values of the implants according to the biodegradation (FIG. 8C) respectively.

FIGS. 9A and 9B show the results of performing immunofluorescence staining on the tissues obtained after transplanting the meshes into the acellular allogeneic dermis and the animal dermis (FIG. 9A) in order to verify the distribution and number of blood vessels (arterioles) inside the meshes depending on whether or not the meshes are coated with collagen, and the results of quantifying the numbers of blood vessels (FIG. 9B) respectively.

FIG. 10 is a photograph showing the macroscopic shape of a structure in which a single collagen sponge and a polymer mesh are directly printed and bonded.

FIG. 11 is electron micrographs showing a fine shape in which a polymer mesh is printed on a collagen sponge and bonded thereto.

FIG. 12 is diagrams showing the results of analyzing the physical properties of a collagen sponge and a structure in which a polymer mesh is printed on the collagen sponge and bonded thereto.

### MODE FOR INVENTION

Hereinafter, the present disclosure will be described in more detail through examples. These examples are only for illustrating the present disclosure in more detail, and it will be obvious to those skilled in the art that the scope of the present disclosure is not limited by these examples according to the subject matter of the present disclosure.

### EXAMPLE

### Example 1: Preparation of Biodegradable Polymer Mesh

### 1-1. Fabrication of Polymer Mesh

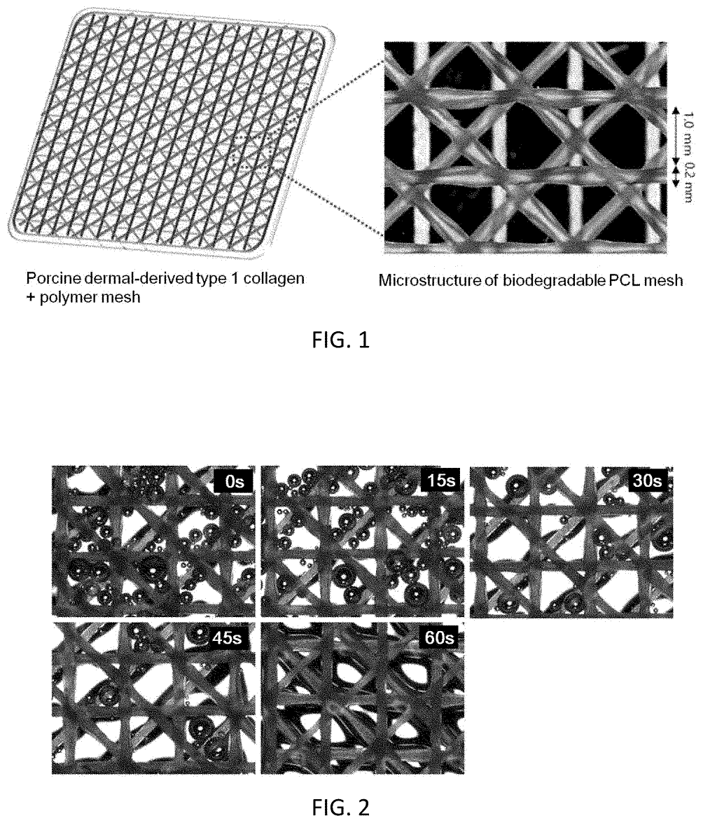

A three-dimensional printer (Biobots, USA) was used in order to prepare a three-dimensional polymer structure, and the three-dimensional printing technique can easily adjust the size of a mesh depending on conditions such as nozzle diameter, temperature, discharge pressure, and nozzle movement speed. The present inventors selected a mesh form including strands each having a diameter of 0.2 mm and a spacing of 1.0 mm between the strands as the design that can most stably support the damaged ligament and abdominal wall (FIG. 1), and polycaprolactone (Sigma Aldrich, USA) was used as a raw material polymer.

In order to fabricate a polymer mesh, the diameter of the nozzle was set to 0.1 to 0.5 mm, the nozzle temperature was set to 80 to 90° C, the discharge pressure was set to 50 to 100 psi, and the nozzle movement speed was set to 2 to 5 mm/s. The polycaprolactone mesh prepared under these conditions was processed into a circular specimen having a diameter of 1.5 cm through a punching operation, washed with 70% ethanol for about 30 minutes in order to remove foreign substances, and then dried at room temperature for 2 hours.

### 1-2. Coating of Polymer Mesh with Collagen

The surface of the mesh was coated with collagen in order to impart biocompatibility to the polycaprolactone mesh prepared by three-dimensional printing. In order to homogeneously coat collagen, the present inventors introduced a pretreatment process that imparts hydrophilicity by subjecting polycaprolactone with strong hydrophobicity before coating to surface treatment using plasma. First of all, a collagen solution was prepared by dissolving atelocollagen (type 1, medical device grade, Dalim Tissen Co., Ltd., Korea) extracted from porcine dermis in 0.5 M acetic acid at a concentration of 0.5% at 4° C for 12 hours.

### Exploration of Optimal Plasma Treatment Time

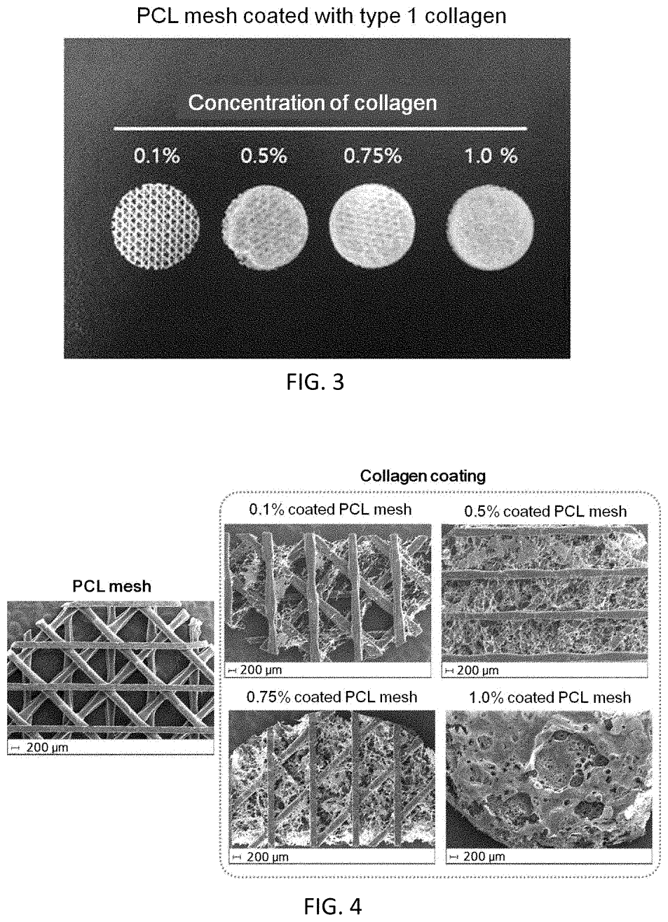

In order to select the optimal plasma treatment time for the most efficient collagen coating, the caprolactone mesh having been dried after washing was placed on a slide glass, and then treated using a plasma surface treatment machine (PDC-32G Plasma Cleaner, Harrick Plasma, USA) for 0, 15, 30, 45, and 60 seconds under medium vacuum conditions of 1.0 to 0.1 Torr. After the surface treatment process, 250 μl of a collagen solution per specimen was put therein to coat the mesh surface with collagen at 4° C for 30 minutes, and the collagen-coated mesh was observed with an optical microscope (EVOS® XL Core Cell Imaging System, Thermo Fisher scientific, USA) (FIG. 2). As shown in FIG. 2, it could be observed that the collagen-coated polycaprolactone mesh that had not been subjected to plasma surface treatment had not only an uneven collagen coating due to the strong hydrophobicity of the surface thereof, but also many bubbles generated on the surface thereof. It could be observed that, as the plasma treatment time was gradually increased at intervals of 15 seconds, the bubbles tended to decrease. When plasma was applied for 60 seconds, it could be observed that a uniform collagen coating film was formed on the surface of the mesh.

### Exploration of Optimal Collagen Concentration

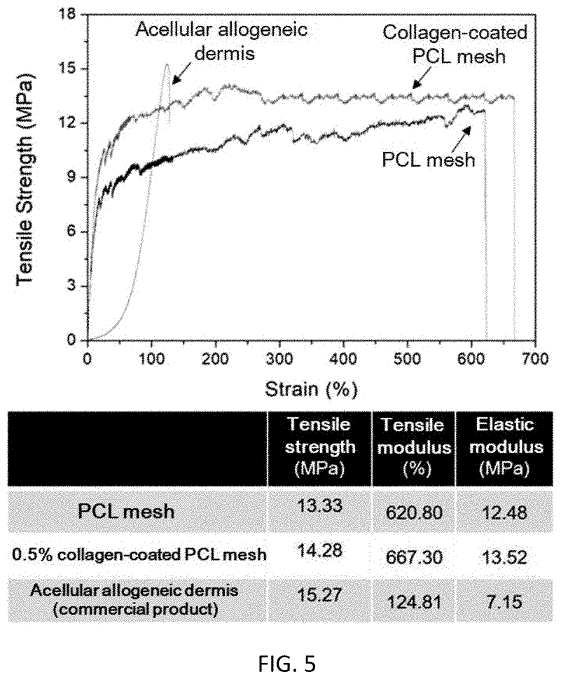

Thereafter, the present inventors tried to evaluate the optimal concentration of collagen with which the surface is coated by considering physical properties, biocompatibility, etc. that the polymer mesh should have as a human body insert. For this, collagen solutions were prepared by dissolving atelocollagen in 0.5 M acetic acid at various concentrations (0.1, 0.5, 0.75, and 1.0%) at 4° C for 12 hours, plasma surface treatment was performed for 60 seconds, and then 250 μl of the collagen solution was put into each of the mesh specimens to carry out a coating operation at 4° C for 30 minutes. Each sample that had been subjected to the coating operation was cooled to −70° C for 12 hours and then dried using a freeze dryer (FreeZone 12 plus, Labconco, USA) for 24 hours in order to create a porous surface structure of collagen with which the surface thereof is coated. Thereafter, a neutralization operation was performed in order to remove acetic acid present in the form of a salt inside freeze-dried collagen. For this, after the specimen that had been freeze-dried was washed 4 times for 15 minutes using anhydrous alcohol (ethanol absolute, Merck KGaA, Germany), 0.5 M NaOH (Duksan General Science, Korea) was dissolved in 70% ethanol, and then the neutralization operation of acetic acid was performed 4 times for 15 minutes. Thereafter, in order to remove the residual amount of NaOH present in the specimen, the collagen-coated mesh was sequentially washed 4 times for 15 minutes using 50% ethanol, 30% ethanol, and tertiary distilled water. After the collagen-coated mesh that had been washed was cooled to −70° C for 12 hours, and dried using a freeze dryer for 24 hours as mentioned above, images were obtained using a digital camera (EOS 500D, Canon, Japan) (FIG. 3). As a result, macroscopic shapes in which collagen blocked pores while being laminated in the form of a sponge on the mesh surface were observed from the group in which the meshes were coated with collagen at a concentration of 0.5% or more, this phenomenon worsened as the concentration of collagen increased, but the shapes of the meshes coated with collagen at a 0.1% concentration were completely preserved.

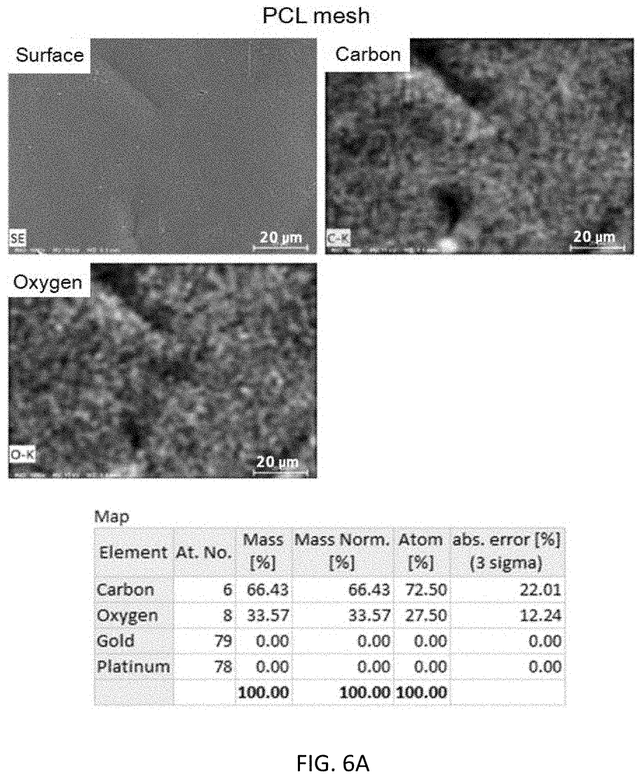

Next, the surface shapes of the collagen-coated meshes were observed using an electron microscope (FE-SEM, MERLIN, Zeiss, Germany) in order to observe the micro-shapes of the collagen-coated meshes (FIG. 4). As a result, when the meshes were not coated with collagen, it was confirmed that each polycaprolactone strand had a diameter of about 200 μm as originally designed. In the collagen-coated specimens, as the collagen concentration increased to 0.1 to 0.75%, the pores of collagen formed by freeze drying were decreased from about 500 μm to 20 μm. However, in the specimen coated with 1.0% collagen, the entire mesh surface was covered with collagen so that the pores could not be observed. The porous structure of collagen thus formed is a structure useful for initial cell attachment and the formation of blood vessels into the mesh when inserted into the human body. Judging from the results of surface observation using an electron microscope, it was determined that the mesh coated with 0.5% collagen, which was confirmed to have pores of about 150 to 300 μm, would be most suitable as a biodegradable mesh for transplantation.

### Example 2: Analysis of Biodegradable Mesh Properties

### 2-1. Analysis of Physical Strength of Biodegradable Meshes

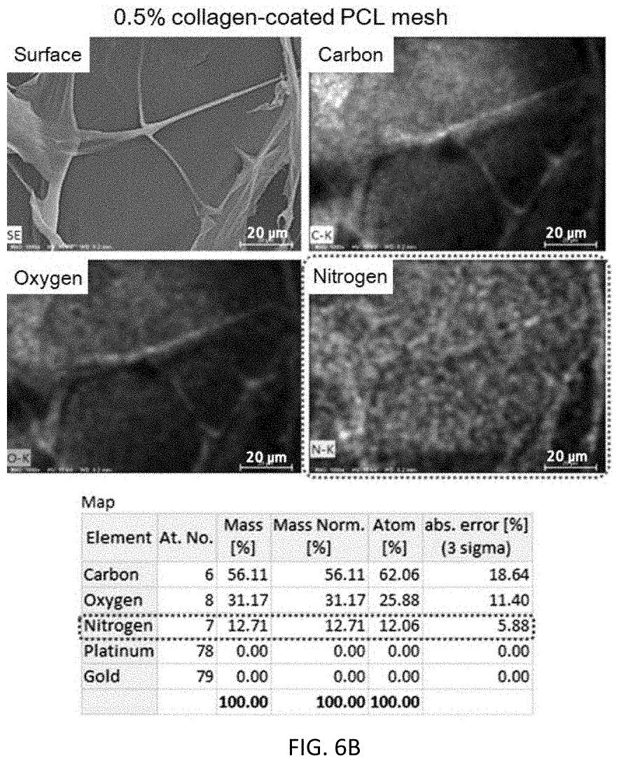

The tensile strength values were measured in order to analyze the physical strength values of the biodegradable meshes for transplantation fabricated in the present disclosure. In order to secure analysis results with higher reliability, acellular allogeneic dermis (CG Derm, Korea) commercially available as a ready-made article for the purpose of reconstruction of soft tissues of the human body was set as a comparison group, and the strength thereof was compared with that of the mesh for transplantation developed by this research team. To this end, after each specimen was processed into a 1 cm×5 cm rectangle and soaked in physiological saline for 30 minutes, the tensile strength was measured while the specimen was pulled at a speed of 1 mm per second using an all-around test analyzer (Universal Testing Systems, Instron 3360, USA). As a result, acellular allogeneic dermis that was the ready-made article showed a lower elastic force than the mesh for transplantation according to the present disclosure until it showed a tensile modulus of 50%, but showed the highest tensile strength of 15.27 MPa at the point of showing a tensile modulus of 124% (FIG. 5). On the other hand, it could be confirmed that the elastic modulus and tensile modulus of the mesh for transplantation according to the present disclosure were 2 times and 5 times higher than those of acellular allogeneic dermis, respectively, so that the elastic restoring force thereof was remarkably excellent. This high elastic restoring force of the mesh for transplantation according to the present disclosure shows that the mesh has very excellent properties as a human body insert for providing physical reinforcement to the ligaments, abdominal wall region, or the like.

### 2-2. Qualitative Analysis of Biodegradable Meshes

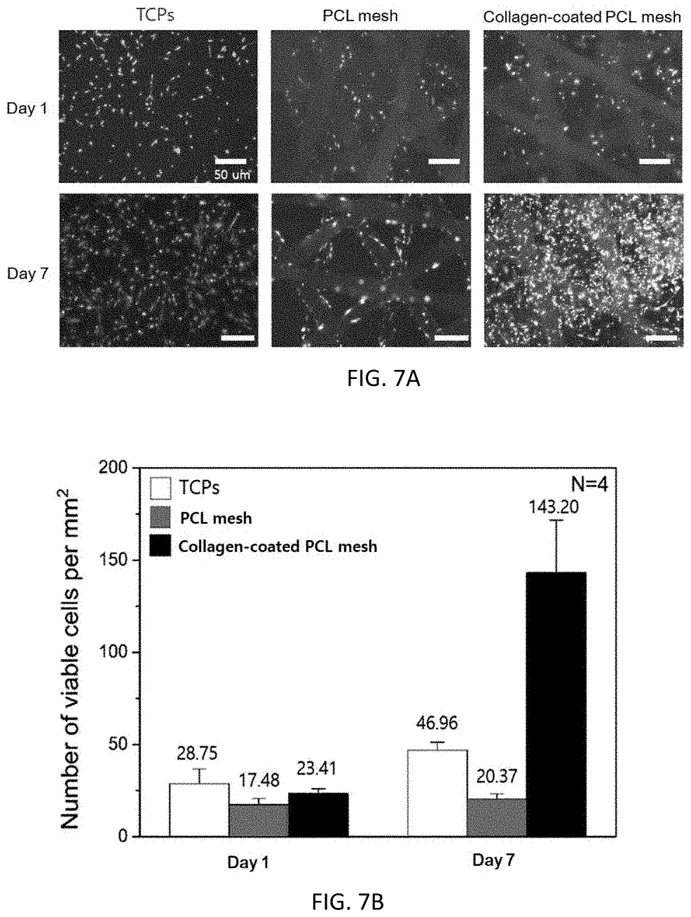

Elements present on the surface of the mesh for transplantation according to the present disclosure depending on whether or not the mesh is coated with collagen were analyzed using Energy Dispersive X-Ray Spectroscopy (EDS) (EDAX, USA). As a result, it could be confirmed that only carbon and oxygen components were detected in the polycaprolactone mesh that was not coated with collagen, whereas nitrogen in the peptide was detected in the specimen whose surface was coated with collagen so that 12.71% of the nitrogen element in the total element ratio was existed (FIG. 6).

### 2-3. Cellular Reactivity of Biodegradable Mesh

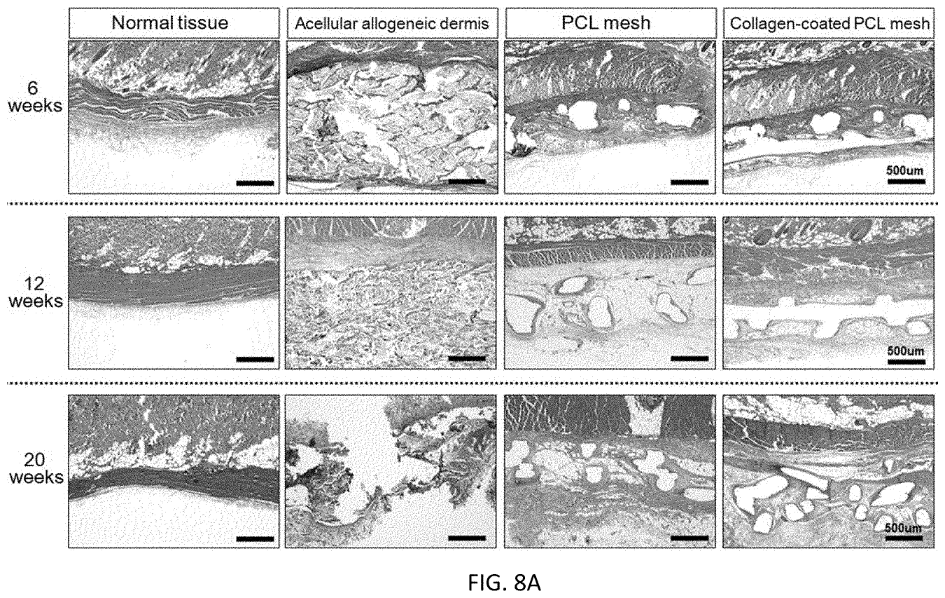

Human dermal-derived fibroblasts (LONZA, USA) were cultured on the mesh surface in order to evaluate the reactivity between the collagen-coated mesh for transplantation and cells in an in vitro environment. After the previously prepared circular specimens having a diameter of 1.5 cm were placed on a 24-well tissue culture plate (TCP, Corning, USA), 70% ethanol was put thereinto, and a sterilization operation was performed for 30 minutes under a UV lamp. Thereafter, 50,000 fibroblasts (passage number 4) were seeded in each specimen and cells were seeded even in TCP as a control group, and then each of the fibroblasts was cultured at 37° C under 5% carbon dioxide conditions using a medium in which 10 v/v % Fetal bovine serum (FBS) (Gibco, USA) and 1 v/v % antibiotic (Gibco, USA) were mixed with Dulbecco's Modified Eagle Medium (DMEM) (low glucose, Gibco, USA) for 7 days. At this time, in order to analyze behaviors of the cells, the survival/proliferation behaviors of the cells were comparatively analyzed by performing live and dead assays (Thermo Fisher Scientific, USA) on the 1st and 7th days after the start of culture. To this end, after each specimen was washed three times with a phosphate buffer solution (PBS, Gibco, USA) at the end of the culture, calcein AM and ethidium homodimer-1 (EthD-1) in the live and dead assay kit were diluted to concentrations of 2 μM and 4 μM respectively, the diluted solutions were put into each specimen, and the cells were stained at room temperature for 30 minutes, and then the stained cells were observed using a confocal fluorescence microscope (LSM700, Zeiss, Germany) (FIG. 7A), and the observed stained cells were quantitatively analyzed (FIG. 7B). As a result, on the first day of culture, 20 to 30 cells with high activity per unit area (1 mm²) were observed in all three groups of specimens, and there seemed no difference between the groups. However, on the 7th day of culture, it was observed that the number of cells per unit area in the collagen-coated mesh group for transplantation was 7 times higher than that of the collagen-uncoated group and about 3 times higher than that of TCP, suggesting that obvious cellular response results could be observed depending on the presence or absence of collagen. This appears to be the result obtained since the porous collagen structure existing between the meshes provides enough space for the cells to attach and proliferate. Accordingly, it can be seen that when the scaffold according to the present disclosure is inserted into the human body after tissue dissection, the attachment of various cells including the initial fibroblasts and the formation of blood vessels into the mesh can be efficiently induced.

### Example 3: Biological Safety of Biodegradable Meshes

### 3-1. Inflammatory Responses and Biodegradation Behaviors of Biodegradable Meshes

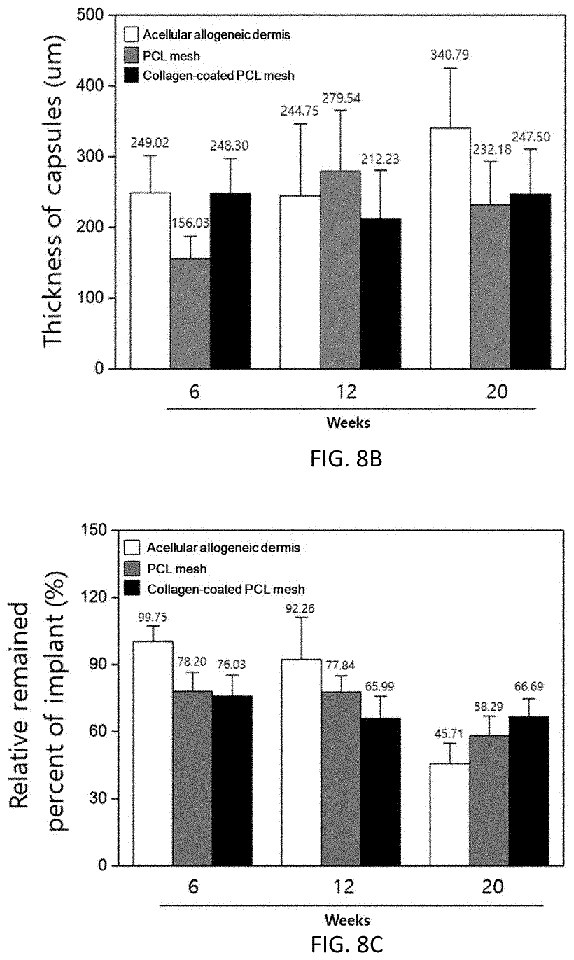

In order to evaluate the inflammatory responses and biodegradation behaviors depending on whether or not the mesh for transplantation according to the present disclosure is coated with collagen, after transplanting meshes together with acellular allogeneic dermis (thickness: 1.5 mm, MegaDerm, L&C Bio, Korea) on the dorsal skin of Sprague Dawley (SD) rats (6 weeks old, male N=4, Orient Bio, Korea) and euthanizing the rats at week 6, week 12, and week 20 to collect tissues, the collected tissues were stained with Masson's Trichrome (Sigma Aldrich, USA) to observe the cross sections of the tissues with an optical microscope (CX43, Olympus, Tokyo, Japan) (FIG. 8A). Further, the inflammatory responses of the transplantation periphery (FIG. 8B) and the biodegradation degrees of the implants (FIG. 8C) were analyzed.

As shown in FIG. 8A, the epidermis, dermis, and subcutaneous tissues of the skin were all clearly observed at the interfaces of normal tissues for 20 weeks, and it could be observed in the groups into which the meshes and acellular allogeneic dermis were inserted that the implants were inserted under the dermal tissues without moving the position. However, unlike the acellular allogeneic dermis, it could be confirmed that the tissues were filled between the porous structures formed by the meshes in all groups in which the meshes were inserted, but in the acellular allogeneic dermis, films were formed thick due to excessive inflammatory responses at week 20, and a delamination phenomenon from the tissues was observed.

In order to analyze the previously observed inflammatory responses, the thickness values of the films formed on the implant periphery were measured (FIG. 8B). As a result of the measurement, it could be observed that the acellular allogeneic dermis formed a film of about 250 μm similar to that of the collagen-coated mesh in the 6th week of transplantation, and as it progressed to the 12th week, a 200 to 280 μm film was formed in all groups of implants so that similar numerical values could be confirmed. However, it could be confirmed that a thick film of about 340 μm was formed in the acellular allogeneic dermis group at week 20, whereas the film of 250 μm, similar to that of week 6, was maintained in the mesh group regardless of whether or not the meshes were coated with collagen. Judging from the results of the Masson's Trichrome staining photographs observed at week 20, such results may be inferred as a phenomenon caused by excessive inflammatory responses.

Next, in order to compare the biodegradation behaviors of the implants, changes in the thickness of each of the implants for 20 weeks were measured (FIG. 8C). At week 6, 99% of the acellular allogeneic dermis remained close to the thickness of the first inserted implant, but about 78% of the acellular allogeneic dermis remained in the mesh group, confirming a decrease in the thickness of about 22%. This trend was maintained until week 12, and the thickness of the acellular allogeneic dermis was maintained at 92%, but the thicknesses of the meshes were maintained at about 70%. However, as a rapid decrease in the thickness of the acellular allogeneic dermis occurred at week 20 compared to week 12 so that only about 45% of the original thickness remained, it could be confirmed that rapid biodegradation occurred within 8 weeks. As shown in FIG. 8B, it can be seen from such results that the thickest film was formed on the transplantation periphery due to an inflammatory response according to the rapid biodegradation of the acellular allogeneic dermis inserted into the tissues at week 20.

### 3-2. Blood Vessel Formation Ability Inside the Biodegradable Mesh

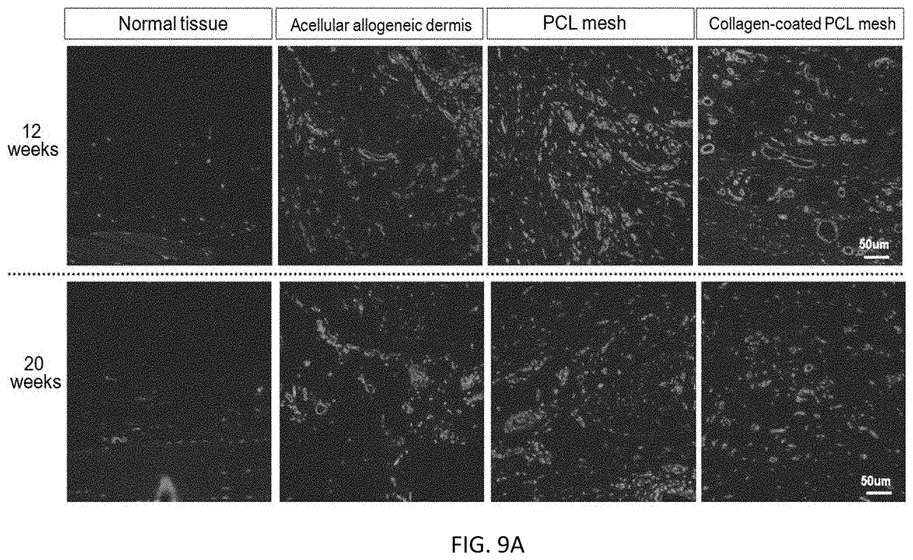

After performing immunostaining on the previously collected tissues in order to evaluate the ability to induce angiogenesis depending on whether or not the mesh for transplantation according to the present disclosure is coated with collagen, staining the cell nucleuses with 4′,6-diamidino-2-phenylindole (DAPI, Blue signal, Sigma Aldrich, USA), and staining vascular endothelial cells with CD31 (Red signal, Thermo Fisher Scientific, Waltham, Mass., USA), the stained cell nucleuses and vascular endothelial cells were observed using a confocal microscope (LSM700, Carl Zeiss, Oberkochen, Germany) (FIG. 9A), and the numbers of blood vessels (arterioles) per area were comparatively quantified (FIG. 9B).

As can be seen from the fluorescence micrographs of FIG. 9A, uneven distribution of blood vessels was observed within the acellular allogeneic dermis over the week 12 and week 20, whereas it could be confirmed that the blood vessels were uniformly distributed to the inside of the meshes regardless of whether or not the meshes were coated with collagen in the tissues into which the meshes were inserted. Such a phenomenon may be inferred from the local distribution of blood vessels due to the reduction of the cross-sectional area caused by the acellular allogeneic dermis, which is rapidly decomposed and decreases in thickness at week 20.

Arterioles of the SD rats are known to have a diameter of 20 to 40 μm, and the numbers of blood vessels satisfying the diameter conditions of arterioles per unit area (mm²) was quantified through immunofluorescence staining (FIG. 9B). At week 12 after implant insertion, about 16 similar blood vessels were observed in the acellular allogeneic dermis and meshes, whereas it was confirmed that about 23 blood vessels, which are 40% more than the acellular allogeneic dermis and meshes, were distributed inside the collagen-coated mesh. This trend was maintained by week 20, confirming that the collagen coating actively induced the formation of blood vessels into the meshes.

### Example 4: Preparation and Property Analysis of Collagen Sponge-Polymer Mesh Conjugate

### 4-1. Collagen Sponge Fabrication

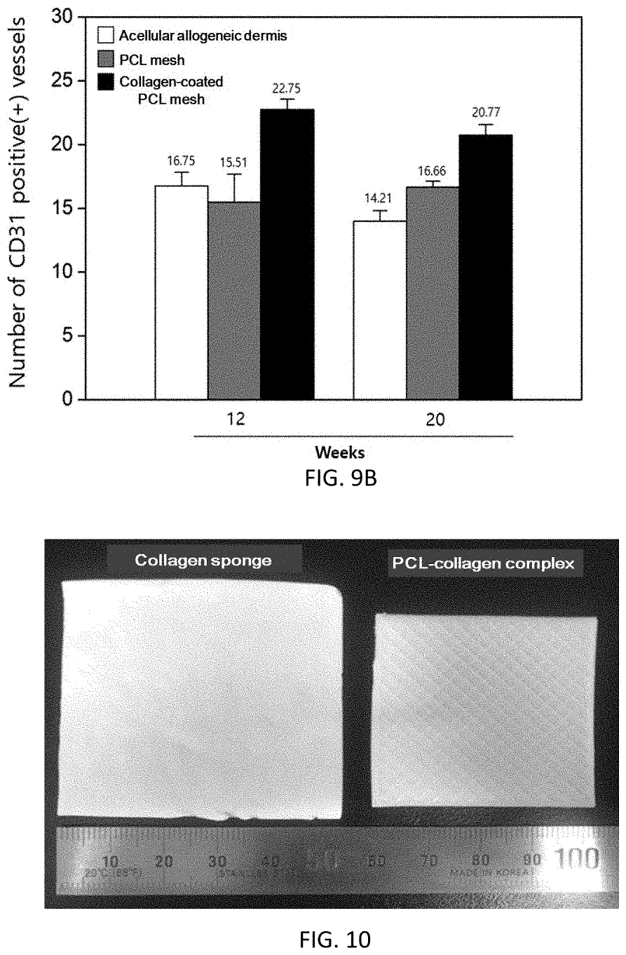

As another aspect of the present disclosure, the present inventors dissolved atelocollagen (Type 1, medical device grade, Dalim Tissen Co., Ltd, Korea) extracted from porcine dermis in 0.5M acetic acid at a concentration of 3.0% by weight in order to fabricate a collagen-containing sponge bonded with a polymer mesh. Thereafter, after putting the dissolved atelocollagen in a brass mold, the brass mold was immersed in liquid nitrogen (−196° C) to freeze, and then freeze-dried for 24 hours according to the method described above in Example 1. Thereafter, the dried collagen sponge was subjected to dehydrothermal treatment (DHT) in an oven at 120° C for 24 hours to prepare a collagen sponge (FIG. 10).

### 4-2. Preparation of Conjugate of Collagen Sponge Polymer Mesh through Three-Dimensional Printing

A PCL-collagen conjugate was fabricated by fixing the sponge to a three-dimensional printing stage in order to reinforce the physical properties of the prepared collagen sponge, and directly printing PCL on the sponge in a mesh form including strands each having a diameter of 0.4 mm and a spacing between the strands of 2.0 mm under the printing conditions applied for polymer mesh fabrication in Example 1 (FIG. 10).

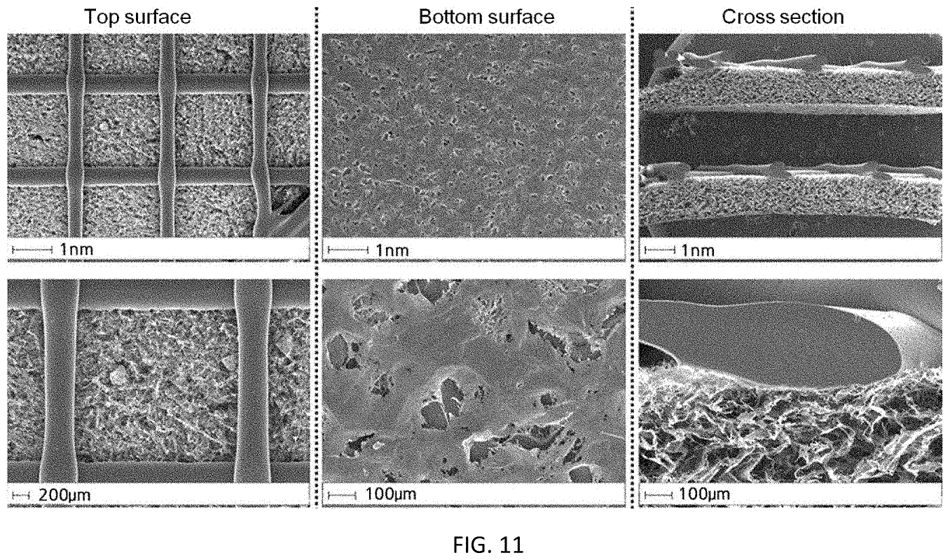

Next, the surface shapes and cross-sectional shapes of the mesh structure in which PCL was bonded onto the collagen sponge through three-dimensional printing were observed using an electron microscope (FIG. 11). As a result, pores of 20 to 200 μm were formed on the surface of the collagen sponge, and it was confirmed through cross-sectional observation that the printed PCL and collagen formed a stably bonded structure.

### 4-3. Analysis of Physical Strengths of Collagen Sponge-Polymer Mesh Conjugate

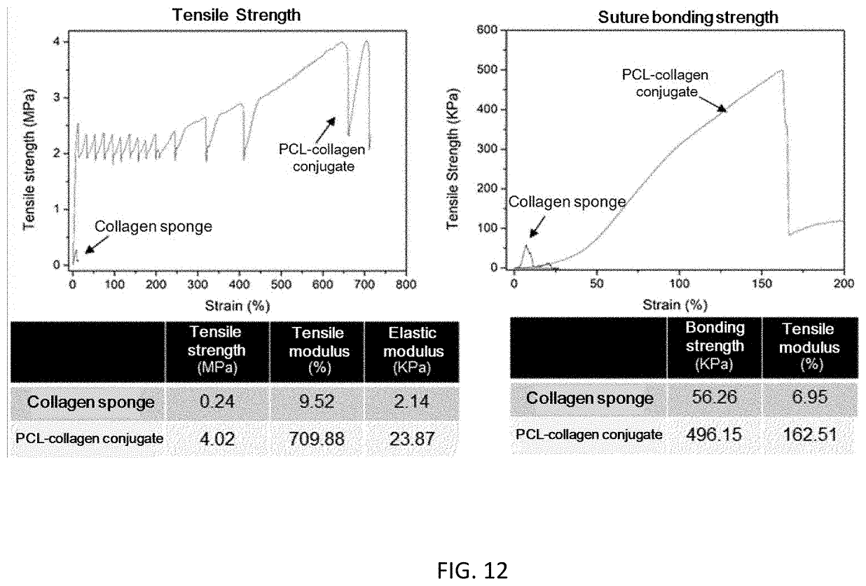

In order to compare and analyze the physical strengths of the prepared collagen sponge-polymer mesh conjugate, tensile strengths, and bonding strengths with a stitching fiber used when fixing the human body were respectively measured. As can be seen from the tensile strength measurement result of FIG. 12, it could be seen that the tensile strength of the conjugate according to the present disclosure to which the PCL mesh was bonded was about 20 times higher than that of the simple collagen sponge, and the tensile modulus thereof was also about 70 times superior to that of the simple collagen sponge, and it could be confirmed that the elastic modulus of the conjugate according to the present disclosure to which the PCL mesh was bonded was also about 10 times higher than that of the simple collagen sponge. Next, as a result of passing the stitching fiber through the collagen sponge and the collagen sponge-PCL mesh conjugate according to the present disclosure respectively and analyzing the bonding strengths with the stitching fiber, it was observed that the strengths remarkably increased from about 56.26 KPa to 496.15 KPa compared to the simple collagen sponge. Through these results, it could be confirmed that the conjugate according to the present disclosure in which the PCL polymer was bonded onto the collagen sponge provides a stable fixation function within the human body while dramatically improving the physical properties of the existing collagen sponge.

As the specific parts of the present disclosure have been described in detail above, these specific descriptions are only preferred embodiments for those of ordinary skill in the art, and it is clear that the scope of the present disclosure is not limited thereto. Accordingly, the substantial scope of the present disclosure will be defined by the appended claims and their equivalents.

1. A method for preparing a porous scaffold, comprising:
(a) producing a polymer mesh having pores with an area of 0.1 to 0.5 mm² and strands each having a diameter of 0.1 to 0.3 mm from a solution of a first polymer; and
(b) coating the surface of the produced polymer mesh with a solution of a second polymer having biocompatibility.

2. The method of claim 1, wherein the first polymer is selected from the group consisting of polycaprolactone (PCL), poly(L-lactic acid) (PLLA), poly(glycolic acid) (PGA), poly(lactic-co-glycolic acid) (PLGA), poly(L-lactide-co-ε-caprolactone) (LCL), and combinations thereof.

3. (canceled)

4. The method of claim 1, wherein the second polymer having biocompatibility is collagen.

5. The method of claim 4, wherein the collagen solution has a concentration of 0.2 to 0.8% (v/v).

6. The method of claim 1, further comprising performing plasma treatment on a surface of the polymer mesh between the step (a) and the step (b).

7. The method of claim 6, wherein the plasma treatment is performed for 45 to 90 seconds.

8. A porous scaffold comprising:
(a) a first polymer mesh having pores with an area of 0.1 to 0.5 mm² and strands each having a diameter of 0.1 to 0.3 mm; and
(b) a second polymer having biocompatibility with which the surface of the first polymer mesh is coated.

9. The porous scaffold of claim 8, wherein the first polymer is selected from the group consisting of polycaprolactone (PCL), poly(L-lactic acid) (PLLA), poly(glycolic acid) (PGA), poly(lactic-co-glycolic acid) (PLGA), poly(L-lactide-co-ε-caprolactone) (LCL), and combinations thereof.

10. (canceled)

11. The porous scaffold of claim 8, wherein the second polymer having biocompatibility is collagen.

12. A support composition for human body transplantation comprising the porous scaffold of claim 8.

13. The support composition of claim 12, wherein the support composition is used for ligament reconstruction, craniofacial reconstruction, maxillofacial reconstruction, tissue reconstruction after removal of melanoma or head and neck cancer, chest wall reconstruction, delayed burn reconstruction, or abdominal wall reinforcement.

14. A method for tissue reconstruction comprising transplanting the support composition of claim 12 in vivo.

15. A method for preparing a dual structure porous scaffold comprising embossing a first polymer having biocompatibility into a mesh form on the surface of a support containing a second polymer having biocompatibility.

16. The method of claim 15, wherein the second polymer having biocompatibility is collagen.

17. The method of claim 16, wherein the support containing collagen is a collagen sponge.

18. The method of claim 15, wherein the first polymer is selected from the group consisting of polycaprolactone (PCL), poly(L-lactic acid) (PLLA), poly(glycolic acid) (PGA), poly(lactic-co-glycolic acid) (PLGA), poly(L-lactide-co-ε-caprolactone) (LCL), and combinations thereof.

19. (canceled)

20. The method of claim 15, wherein the embossing is performed by outputting the first polymer in a mesh form using a three-dimensional printer on the surface of the second polymer-containing support.

21. The method of claim 15, wherein the mesh form includes strands each having a diameter of 0.3 to 0.5 mm and a spacing between the strands of 0.1 to 0.3 mm.

22. A dual structure porous scaffold including the following:
(a) a support containing a second polymer having biocompatibility; and
(b) a first polymer mesh which is bonded to the surface of the support and has biocompatibility.

23. A method for tissue reconstruction comprising transplanting the dual structure porous scaffold of claim 22 in vivo.
